(19) United States
(12) Patent Application Publication
(10) Pub. No.: US 2017/0367574 A1
(43) Pub. Date: Dec. 28, 2017

(54) EYE CYTOMETER FOR CONTINUOUS HEALTH MONITORING

(71) Applicant: Verily Life Sciences LLC, Mountain View, CA (US)

(72) Inventors: Chinmay Belthangady, San Francisco, CA (US); Seung Ah Lee, Menlo Park, CA (US); John D. Perreault, Mountain View, WA (US); Jennifer Sy-En Han, Palo Alto, CA (US); Cheng-Hsun Wu, Mountain View, CA (US); Mark Lee, Los Altos Hills, CA (US)

(21) Appl. No.: 15/497,015

(22) Filed: Apr. 25, 2017

Related U.S. Application Data
(60) Provisional application No. 62/354,150, filed on Jun. 24, 2016.

(51) Int. Cl. A61B 3/14; A61B 5/1455; A61B 5/145; A61B 3/113; G01N 15/14

(52) U.S. Cl. CPC A61B 3/145; A61B 3/113; G01N 15/1434; A61B 5/14535; A61B 5/1455; G01N 2015/144

(57) ABSTRACT

Systems and methods are provided for cytometric measurement of blood cells traversing microvasculature single-file in the eye of a subject. A miniature imaging device, having cellular resolution, records image data that can be rendered into a microcirculation time sequence and analyzed to provide useful biological information.

### CROSS REFERENCE TO RELATED APPLICATION

This application claims the benefit of U.S. Provisional Application No. 62/354,150 filed on Jun. 24, 2016, the contents of which are incorporated herein by reference.

### BACKGROUND

The present disclosure generally relates to biophysical systems and methods, and solutions in the field of cytometry. More particularly, and without limitation, the present disclosure includes embodiments incorporating small-form-factor or miniaturized devices for imaging parts of the eye and enabling early disease detection and continuous health monitoring.

Examination of the eyes is commonplace for the evaluation of optical and ophthalmological health. Tests commonly used range from the standard visual acuity tests using a standard eye chart and the cover chart, to refraction tests using a phoropter. Currently, fundus cameras represent the standard technology used for retinal imaging in ophthalmology departments. However, these cameras are bulky and expensive, and not practical in a general care setting. Medical clinics, including in rural or developing areas, also may not have the resources or expertise to operate highly specialized ophthalmic equipment. Retinal scanning techniques are also known for identification purposes, and may also be used for the diagnosis of pathologies that affect the eyes, for example diabetes, malaria, AIDS, syphilis, malaria, chicken pox, Lyme disease, leukemia, lymphoma, sickle cell anemia, multiple sclerosis, congestive heart failure, atherosclerosis and hypercholesterolemia. Other health conditions, such as pregnancy, also affect the eyes.

At present, eye examinations are primarily performed in an ophthalmologist's office or other specialized point-of-care location. Instruments used for imaging the eye generally cannot be used by patients at home. Recently, attempts have been made to reduce the size and expense of retinal imagers, for example using smartphone technology, to provide higher quality eye care. Such miniaturized devices image the retina with large fields of view and low resolution to gather structural information that can aid in diagnosing diseases of the eye. However, these miniaturized devices have a number of limitations, including in field of vision, image quality and stabilization.

In view of the above and other factors, conventional eye measurement devices suffer from numerous drawbacks. These drawbacks are especially acute in situations where a patient does not have access to an ophthalmological facility, or where miniaturized instruments cannot obtain images of the quality level necessary to overcome the presentation limitations of the subject.

### SUMMARY

The disclosed embodiments include systems and methods for imaging parts of the eye using a miniaturized device with cellular resolution corresponding to a red blood cell, and at multiple optical wavelengths, so that parameters that affect the diagnosis and prognosis of various diseases that benefit from continuous monitoring can be quantitatively measured.

According to illustrative embodiments of the present disclosure, a system for single-file cytometry of the microvasculature of the eye of a subject is described, the system including an imaging device having imaging optics with at least one lens and a sensor, the lens and sensor having a focal length permitting cellular resolution of intraocular structures from outside the eye, and an optics controller coupled to at least one illumination source, and an image processor, the image processor coupled to the sensor of the imaging optics, and the image processor having a video encoder that creates a microcirculation time sequence showing the transit of cells through the microvasculature of the eye.

According to a further illustrative embodiment of the present disclosure, a method for eye cytometry is described, comprising the steps of capturing high-frame-rate video images of the microvasculature within the eye of a subject; registered the high-frame-rate video images; filtering the high-frame-rate video images to remove noise and artifacts; segmenting the high-frame-rate video images; and creating a microcirculation time sequence from the high-frame-rate video data showing the transit of cells through the microvasculature over a period of time.

Additional features and advantages of the disclosed embodiments will be set forth in part in the description that follows, and in part will be obvious from the description, or may be learned by practice of the disclosed embodiments. The features and advantages of the disclosed embodiments will be realized and attained by the elements and combinations particularly pointed out in the appended claims.

It is to be understood that both the foregoing general description and the following detailed description are examples and explanatory only and are not restrictive of the disclosed embodiments as claimed.

The accompanying drawings constitute a part of this specification. The drawings illustrate several embodiments of the present disclosure and, together with the description, serve to explain the principles of the disclosed embodiments as set forth in the accompanying claims.

### BRIEF DESCRIPTION OF THE DRAWINGS

FIG. 1 depicts schematically an eye showing the major parts thereof.

FIG. 2 depicts schematically the illumination of a retinal capillary.

FIG. 3A depicts a schematic view of a system for retinal cytometry, consistent with the disclosed embodiments.

FIG. 3B depicts a schematic view of a system for retinal and/or scleral cytometry, consistent with the disclosed embodiments.

FIG. 4A depicts a schematic view of a retinal scanner system, consistent with the disclosed embodiments.

FIG. 4B depicts a schematic view of a retinal scanner system, consistent with the disclosed embodiments.

FIG. 5A depicts a schematic view of a scleral imaging system, consistent with the disclosed embodiments.

FIG. 5B depicts a schematic view of a scleral imaging system, consistent with the disclosed embodiments.

FIG. 6 depicts a flowchart of an example method for cell cytometry, consistent with the disclosed embodiments.

FIG. 7 depicts a schematic of an exemplary processing system, consistent with the disclosed embodiments.

### DETAILED DESCRIPTION

The disclosed embodiments relate to systems and devices allowing for substantially continuous imaging of parts of the eye using a miniaturized device with cellular resolution from 5 microns to 20 microns and at multiple optical wavelengths and/or subwavelengths, so that various parameters used in the diagnosis and prognosis of various diseases can be quantitatively measured. For example, resolution may be 0.1 μm, 1 μm, or 10 μm.

According to embodiments of the present disclosure, principles of reflectance and absorption are used in small vasculature using a wide-field imaging device. Optical interrogation wavelengths are selected to correspond to an analyte under assay, which in the example of hemoglobin is between about 540 and 575 nm for oxygenated hemoglobin and about 555 nm for deoxygenated hemoglobin.

Reference will now be made in detail to embodiments of the present disclosure, examples of which are illustrated in the accompanying drawings. Where possible, the same reference numbers will be used throughout the drawings to refer to the same or like parts.

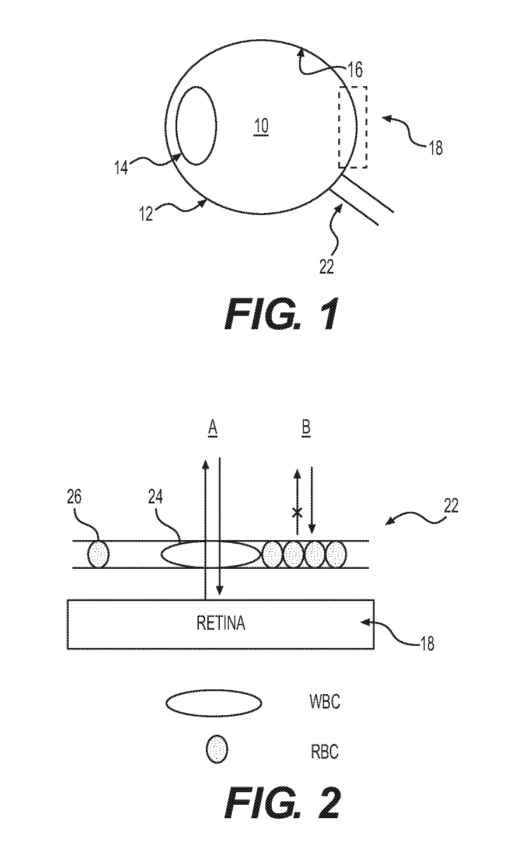

Referring to FIG. 1, an eye 10 is schematically depicted. The more or less globular capsule of eye 10 is formed by sclera 12 and the transparent cornea that covers the iris and pupil. Lens 14 resides behind the cornea, and refracts light toward retina 16, which is the light-sensitive structure on which a visual image is formed. The space between lens 12 and retina 16 is occupied by a clear gel material called the vitreous humor. Foveal avascular region 18 surrounds the fovea centralis at the center of the macula lutea of the retina, and is itself devoid of blood vessels.

Underlying the foveal avascular region 18 on the retina 16 and sclera 12 are present, as shown in FIG. 2, narrow capillaries 22 where blood cells flow past in single file. As further depicted in FIG. 2, single-file flow of blood cells in retinal capillaries can be imaged by illumination at appropriately chosen wavelength(s). Low absorption by leukocytes (WBC) 24 results in high reflection as shown by arrows A. Erythrocytes (RBC) 26 absorb a significant fraction of incident light and trailing RBCs show reduced reflectance as shown by arrows B. Cell counting in a region around the fovea (or sclera) can be used, for example, to estimate their population in the body of an individual and for other purposes described herein.

According to some aspects of the present disclosure, an instrument referred to as an eye cytometer is described. The eye cytometer can be used to record high-resolution video recordings of the single-file flow of blood cells via reflectance microscopy to count the number, shape and size of these blood cells. Advantageously, a noninvasive “blood test” can be performed without drawing blood or labeling cells with exogenous contrast agents, dyes, or tags.

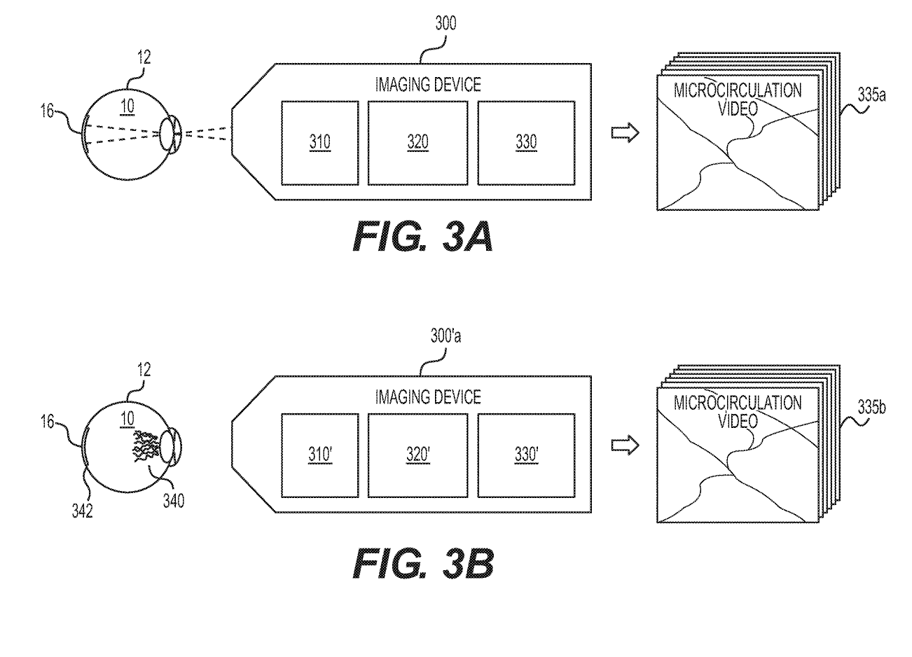

Referring to FIG. 3A, an illustrative embodiment of a retinal cytometer is depicted, consistent with the present disclosure. As discussed above, the eye 10 is enveloped by the sclera 12, with the retina 16 formed on the interior of the rear of the eyeball. In the example embodiment of FIG. 3A, imaging device 300 includes imaging optics 310 including one or more lenses. The lenses may include an objective lens, such as a simple objective, or telephoto or retrophoto objective lens. As further described below, device 300 may also include an optics controller 320 for manipulating the F-number, aperture, focal length, power, and focus of imaging optics 310. In some embodiments, the focal length and aperture are fixed. In still further embodiments, light field imaging is employed to capture an imaging data stack and then focusing is obtained during one or more post-process. As mentioned above, the objective lens can be a simple objective, or telephoto or retrophoto objective. Lenses of imaging optics 310 can vary in terms of their refractive properties. In some embodiments, the lens is formed of a doublet or stack of elements made of optical materials, for example, glass, PMMA, polystyrene, etc. Imaging optics 310 may also include a sensor upon which images from the one or more lens are formed for digitization and further processing. CMOS sensors are an exemplary sensor that can be used in eye cytometers according to the present disclosure. As will be appreciated, sensors can vary in terms of speed, frequency response, etc.

As further shown in FIG. 3A, imaging device 300 also includes optics controller 320 for controlling the exposure of the imaging optics 310, which is related to aperture and exposure (or shutter) time and the amount of available light. The imaging device 300 can be “shutterless,” where the exposure time is controlled electronically. The correct amount of exposure will ensure a quality image, and depends on the imaging sensor being employed. Optics controller 320 measures the amount of available light and calculates the exposure based on current imaging settings, and makes adjustments where necessary to ensure image quality. Optics controller 320 also can control an illumination source to add illumination to the imaging field.

Image processor 330 receives output from the image sensor and processes that output. In an illustrative embodiment, image processor 330 includes a signal processor that performs various operations on image data, for example, demosaicing, auto white balance, noise reduction, filtering, and corrections for lens aberrations and other distortions. Image processor 330 also provides any wavelength adjustments to render light outside of the visual spectrum to wavelengths visible to the human eye, or to false- or hyper-spectral wavelengths. In some embodiments, image processor 330 may also provide feedback to imaging optics 310 for autofocusing, and to optics controller 320 for autoexposure. Further, in some embodiments, image processor 330 includes a video encoder for processing image frames into a microcirculation video format 335a. By way of example, the video encoder can be VP9, H.264/MPEG-4 HDC, or other video format encoder. The imaging device 300 can be used to acquire a microcirculation video 335a of capillaries on the retina 16 of the eye 10, as depicted in FIG. 3A.

In some embodiments, imaging device 300 acquires images of retinal microcirculation using the optics of the eye 10. In some examples, the imaging optics 310 may be focused at infinity space. Optics controller 320 may use focusing and/or autofocusing algorithms to adjust for the optical power and/or aberrations of the eye 10.

Referring to FIG. 3B, an illustrative embodiment of a retinal and/or scleral cytometer is depicted, consistent with the present disclosure. In this embodiment, imaging device 300′a includes imaging optics 310′, optics controller 320′, and image processor 330′, which may be implemented in a similar fashion to the corresponding components (310, 320, and 330) discussed in reference to FIG. 3A. The imaging device 300′a can be used to acquire a microcirculation video 335b of microcirculation sites 342 on the retina 16 of the eye 10, or microcirculation sites (e.g., capillaries, arterioles, venules) 340 on the sclera 12 of the eye 10, as depicted in FIG. 3B. In some examples, imaging optics 310′ may be focused at a fixed distance from the objective lens. Optics controller 320′ may use focusing and/or autofocusing algorithms to adjust for the optics of eye 10 and/or fast movement of eye 10.

For retinal imaging, as shown in FIG. 3A, the lens of the eye 10 may serve as the first optical focusing element. When the eye lens is not accommodated, the image of the retinal capillaries may form at infinity. In some examples, the imaging optics package 310 therefore is designed for infinite conjugate imaging. In contrast, for scleral imaging as shown in FIG. 3B, the first focusing element may be the imaging optics package 310′, since no eye lens is present. In some examples, the imaging optics package 310′ therefore is designed for finite conjugate imaging.

In FIG. 3A, image quality may be affected by the optical power or aberrations of the eye, whereas in FIG. 3B the imaging resolution may be independent of the optics of the eye. In both cases, the imaging system requires focusing/autofocusing schemes. However, because of the small depth of focus of the microscope-like imaging system and the fast movement of the eye, the focusing issue is more acute for the system illustrated in FIG. 3B.

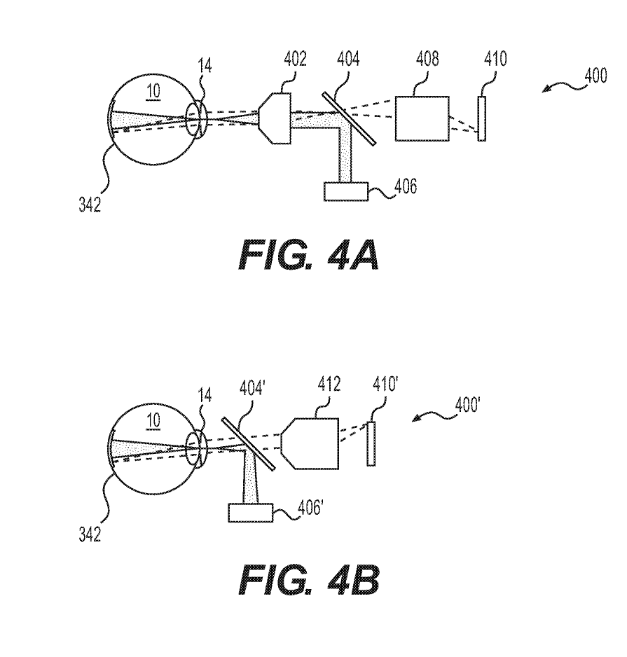

Turning now to FIG. 4A, a schematic view is provided of an illustrative cytometer 400 for imaging retinal microcirculation sites 342 It is to be understood that a similar cytometer system could be employed for imaging scleral microvessels, as previously and subsequently discussed. The optics of cytometer 400 may include an objective lens 402 located in front of beam splitter 404, which deflects light from illumination source 406 into the field of view through the objective lens 402, and permits image information from the microcirculation sites 342 to pass through to the camera lens 408. The camera lens 408 projects the image onto image sensor 410 for digitization and processing, as described above. The digitized optical image can be subsequently recorded to memory and/or transmitted by wired or wireless connection to one or more additional devices (not shown).

Turning now to FIG. 4B, a schematic view is provided of a further illustrative cytometer 400′ for imaging retinal microcirculation sites 342 of the eye 10. It is to be understood that a similar system could be employed for imaging scleral microvessels, as previously and subsequently discussed. The optics of cytometer 400′ may include a beam splitter 404′, which deflects light from illumination source 406′ into the field of view, and permits reflected image data from the microcirculation sites 342 to pass through to the imaging lens 412. The imaging lens 412 projects the image onto image sensor 410′ for digitization and processing, as described above. The digitized optical image can be subsequently recorded to memory and/or transmitted by wired or wireless connection to one or more additional devices (not shown). In the aforementioned embodiment illustrated in FIG. 4A, the objective lens 402 is formed to optically interact with the lens 14 of the eye to form the imaging. Similarly, in the example embodiment illustrated in FIG. 4B, the imaging lens 412 is formed to optically interact with the lens 14 of the eye to form the imaging.

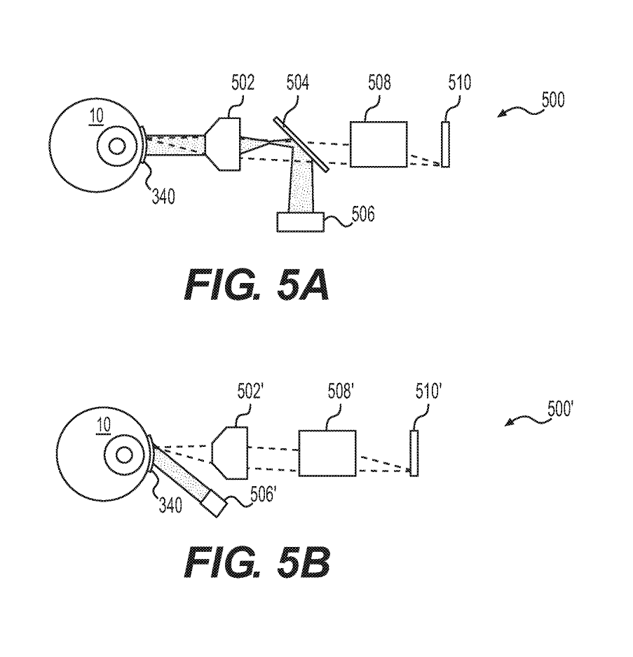

Turning now to FIG. 5A, a schematic view is provided of an illustrative cytometer 500 for imaging scleral microvessels 340 of the eye 10. It is to be understood that a similar system could be employed for imaging retinal microvessels, as previously discussed. The optics of the cytometer can include an objective lens 502 located in front of beam splitter 504, which deflects light from illumination source 506 into the field of view through the objective lens 502, and permits image information from the microvessels 340 to pass through to tube lens 508. The tube lens 508 projects the image onto image sensor 510 for digitization and processing, as described above. The digitized optical image can be subsequently recorded to memory and/or transmitted by wired or wireless connection to one or more additional devices (not shown).

Turning now to FIG. 5B, a schematic view is provided of a further illustrative cytometer 500′ for imaging scleral microvessels 340 of the eye 10. It is to be understood that a similar system could be employed for imaging scleral microvessels, as previously and subsequently discussed. The optics of the cytometer can include an objective lens 502′, which receives reflected image information from the microvessels 340 to pass through to the tube lens 508′. The image field is illuminated by illumination source 506′. The tube lens 508′ projects the image onto image sensor 510′ for digitization and processing, as described above. The digitized optical image can be subsequently recorded to memory and/or transmitted by wired or wireless connection to one or more additional devices (not shown).

The illumination sources 406, 406′, 506 and 506′ described above can be controlled by an optics controller, such as 320 or 320′ shown in FIGS. 3A and 38, respectively. The illumination sources can be broad spectrum, or selected to provide one or more single wavelengths or a range of wavelengths. In an exemplary embodiment, eye vasculature is illuminated with light at certain characteristic wavelengths in the hemoglobin absorption spectrum (e.g., about 420 nm for maximum absorption or the isosbestic wavelengths at about 586 nm or about 808 nm) to measure various parameters. Plural illumination sources can also be used to provide diverse illumination types.

According to one aspect of the present disclosure, erythrocyte counts are performed by direct cell counting, as discussed herein.

According to another aspect of the present disclosure, leukocyte counts are performed by counting gaps in the blood cell flow that are followed by trailing erythrocytes.

According to another aspect of the present disclosure, hemoglobin concentration is calculated by measuring the optical depth of wide capillaries.

According to another aspect of the present disclosure, hematocrit is calculated by measuring the fraction of erythrocytes compared to total flow volume.

According to another aspect of the present disclosure, mean corpuscular volume is calculated by measuring the shapes and sizes of erythrocytes.

According to another aspect of the present disclosure, mean corpuscular hemoglobin concentration is calculated by measuring the optical depth of individual erythrocytes.

According to another aspect of the present disclosure, mean corpuscular hemoglobin is calculated by combining mean corpuscular volume and mean corpuscular hemoglobin concentration.

According to another aspect of the present disclosure, red blood cell distribution width is calculated from mean corpuscular volume.

According to another aspect of the present disclosure, pulse oxygenation is calculated by measuring hemoglobin absorption at two different wavelengths.

According to another aspect of the present disclosure, heart rate is calculated by measuring the intermittency of cell flow velocity.

According to another aspect of the present disclosure, blood pressure is calculated by measuring changes in capillary widths.

According to an aspect of the present disclosure, the noninvasive blood testing apparatus and methods described herein, for example in an illustrative embodiment leukocyte level measurements, can be used for sepsis prevention and monitoring, determining cancer chemotherapy outcomes (e.g. myelotoxicity of chemotherapy, or response of hematologic malignancies to treatment) and guiding chemotherapy dosing, monitoring the health of immunocompromised patients, e.g. those with HIV positive status.

According to other illustrative embodiments, the noninvasive blood testing apparatus and methods described herein may be employed to conduct large-scale longitudinal studies of the immune system, for example during drug trials, monitoring patients with thalassemia or anemia, and early detection of leukemia (including recurrences following definitive therapy).

Many of the diseases and conditions discussed herein are usually not tested for in an ophthalmologist's or optometrist's office where retinal evaluation equipment is available. Further, miniaturized retinal camera apps loaded on smartphones, such as those available from Peek Vision, are not configured for the analyses described herein.

According to other aspects of the invention, the systems and methods of the present disclosure are used in the molecular imaging of endogenous or exogenous optical contrast agents present in the eye with a miniaturized device for early disease detection and monitoring.

According to one aspect of the present disclosure, monitoring bilirubin levels is implemented using reflectance measurements. High levels of bilirubin in the blood causes jaundice which manifests as a characteristic yellowing of the eye. By illuminating the sclera with light at a wavelength corresponding to the absorption maximum of bilirubin (e.g., approximately 450 nm) and measuring the spectrum of the reflected light, the amount of bilirubin in the body can be quantified. This can help in monitoring diseases where jaundice is a symptom for example viral or drug-induced hepatitis, primary or metastatic liver cancers, thalassemia, pancreatic cancer, and sickle cell anemia.

According to one aspect of the present disclosure, detection of fluorescently labeled tumor cells or other circulating targets is implemented by direct ballistic imaging of retinal or scleral vasculature.

According to another aspect of the present disclosure, Fluorescence Lifetime Imaging (FLIM) of endogenous or exogenous contrast agents present in the retina and sclera is implemented.

According to another aspect of the present disclosure, Raman spectroscopy of endogenous or exogenous contrast agents present in the retina and sclera is implemented.

According to another aspect of the present disclosure, hyperspectral reflectance and fluorescence measurements of endogenous or exogenous contrast agents present in the retina and sclera is implemented.

According to a further aspect of the present disclosure, systems and methods are disclosed for the monitoring of motion of the eyeballs and eyelids with a miniaturized device for early detection or monitoring of diseases of neurological origin.

According to one aspect of the present disclosure, a miniaturized device is employed to capture videos of eyelid motion during periods of wakefulness and during sleep (e.g., during the rapid eye movement (REM) phase) and measure the amplitude and frequency of such motion to study correlations with various diseases.

According to a further aspect of the present disclosure, a miniaturized device is employed to measure saccades and microsaccades of the eyeballs and measure their amplitude and frequency. These measurements can be used to follow sleep phases, or abnormality in certain cerebellar diseases.

According to a further aspect of the present disclosure, images captured with the miniaturized eye imagers can also be used to monitor diseases whose symptoms manifest as alterations in the structure of the eye such as diabetic retinopathy, hypercholesterolemia, multiple sclerosis, Alzheimer's (for example via detection of beta amyloid plaques), malaria, atherosclerosis, and cataracts.

In an exemplary embodiment, for retinal imaging, a lens with a long focal length is employed to get sufficient magnification. To maintain optimal F-number (lens speed), the lens will need to have a large diameter. Conventional lenses of adequate lens speed will be bulky and feedback will be slow. For fast feedback, an illustrative embodiment employs a liquid lens instead of a conventional lens as the focusing element. This combination of a PDAF CMOS chip and a liquid lens in an illustrative embodiment advantageously provides long focal length and excellent f-number, along with fast feedback not achievable using conventional lenses.

For retinal imaging, a long focal length lens (which provides magnification) could increase overall package size. To aid miniaturization, an illustrative embodiment uses a telephoto design for the imaging lens, such that the focal length is longer than the physical length of the lens package.

In a further illustrative embodiment, for example for scleral imaging, the objective lens has a short focal length, and a long working distance. Advantageously some illustrative embodiments can employ a retrofocus design.

According to some embodiments, to counter microsaccadic eye motion, contrast detection algorithms can be employed to detect motion blur and provide feedback with a liquid lens that has adaptive optics such as tip-tilt correction. In other embodiments, dynamic focusing with an acoustic lens can be employed so that images in several different focal planes are captured simultaneously and then postprocessed. Alternatively, a lightfield imager could be employed. This approach could be particularly useful for scleral imaging where the surface is convex.

In an exemplary embodiment of the present disclosure, phase contrast imaging can be used, for instance in leukocyte visualization. In this approach, an epigeometry can be employed, and image reconstruction can be realized using the transport-of-intensity method.

In an exemplary embodiment of the present disclosure, autofluorescence for imaging can be used for leukocyte visualization. In this approach, autofluorescence of the background tissue (e.g., sclera) and difference in absorption of the autofluorescence among blood cells can be employed to visualize leukocytes.

In an illustrative embodiment, specular reflection from the cornea and other surfaces can be minimized by polarization filtering where only light polarized orthogonal to the incident light is collected.

Eye cytometers according to the present disclosure can have a widefield or scanning confocal design. A combination binocular design is also possible where wide-field imaging is done in one eye and scanning confocal imaging is done in the other eye.

To correct eye aberrations, which advantageously can positively affect high magnification retinal imaging, the present disclosure provides several approaches that can be implemented in a low-cost platform. In some embodiments, the aberrations in the patient's eyes can be measured at the beginning with a Shack Hartmann (SH) wavefront sensor and a custom phase plate can be made to correct for these errors. A fixation target in the eye cytometer can help align the patient's eye with the phase plate during use. In an exemplary embodiment, the Gerchberg Saxton (GS) algorithm can be used for software-based aberration correction. An initial measurement of the subject's eye aberrations with an SH sensor may be used as a prior for the GS algorithm to enable faster image reconstruction. In an exemplary embodiment, a SH sensor on the eye cytometer is employed to record the eye's aberrations for each frame recorded by the camera sensor. Deconvolving the camera image with the SH image can correct for eye aberrations.

In another embodiment of the present disclosure, full adaptive optics can be used to dynamically correct eye aberrations. To reduce costs, a small form factor LCOS system can be used instead of a deformable mirror.

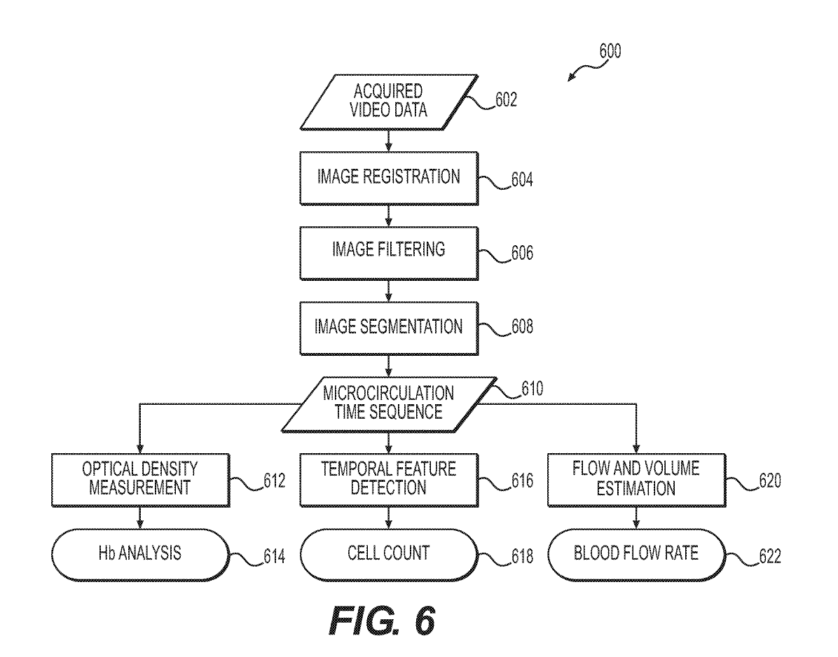

An illustrative method 600 for miniaturized eye cytometry will now be described with reference to FIG. 6. It is to be understood that the various processing steps described herein can be undertaken on the cytometer device itself using an on-board processor, or image data can be transmitted by wired or wireless link to one or more processor(s). At step 602, videos are captured at high frame rates to visualize single cell flow. This approach advantageously increases the data density from the capture of still images in conventional cytometric approaches. Since images need to be in good optical focus throughout the video recording, an autofocusing system may be employed. Frames acquired at step 602 are registered at step 604. Various registrations algorithms can be employed, such as phase correlation, minimizing image differences, motion compensation, and feature tracking. Fast autofocus can be provided by phase detect autofocus (PDAF) CMOS sensors of small form factor in conjunction with contrast detect autofocus. At step 606, image filtering takes place to remove noise and artifacts from the image and in some embodiments can include edge detection, histogram equalization, in-painting, etc. At step 608, image segmentation algorithms are used for identifying cells or other features within the image. Various techniques can be employed for segmentation in various exemplary embodiments, including thresholding, clustering, compression, histogram, and edge detection, dual clustering, region growing and partial differential equation, variational, graph-partitioning, watershed, model-based, multi-scale segmentation, semi-automatic segmentation, and trainable segmentation methods.

During registration of each frame of image data to the previous frame, a certain offset is calculated. In an illustrative embodiment, the offset or shift of each frame to the previous frame is tracked, which over time gives a record of the movement of the eye, assuming the image device is stationary. The frequency and amplitude of microsaccades can be calculated, as well as other eye movements, such as that present in nystagmus.

A microcirculation time sequence is compiled at 610, which can be manually or automatically selected from the video data. This time sequence, showing the transit of cells through the microvasculature over a period of time, can be further analyzed to determine various outputs according to illustrative embodiments of the present disclosure.

Following the compilation of the microcirculation time sequence at step 610, one or more of optional steps can be undertaken, as described below.

In an exemplary embodiment, an optional optical density measurement 612 is calculated, based on the optical reflectance differences between red blood cells and white blood cells as discussed with reference to arrows A and B of FIG. 2. Results from the optical density measurements correlate to hematocrit and can reveal the presence of anemia, as the detected hemoglobin is the iron-containing protein found in all red blood cells that enables RBCs to bind to oxygen in the lungs and carry it to tissues and organs throughout the body. The opposite condition, polycythemia or high hematocrit, can also be determined from optical density.

In an exemplary embodiment, an optional temporal feature detection step 616 is performed to allow a counting of individual cells that pass through the microvasculature per unit time at step 618.

In an exemplary embodiment, an optional flow and volume estimation step 620 is performed to allow blood flow rate to be calculated at step 622.

In certain aspects, the computer-implemented operations and methods described herein may be implemented on a single processor. In other embodiments, these computer-implemented operations and methods may be implemented using one or more processors within a single computing system and/or on one or more processors within separate computing systems in communication over a network. Instructions or code for configuring the processor(s) or computing system(s) may be stored in a computer-readable medium or other memory device.

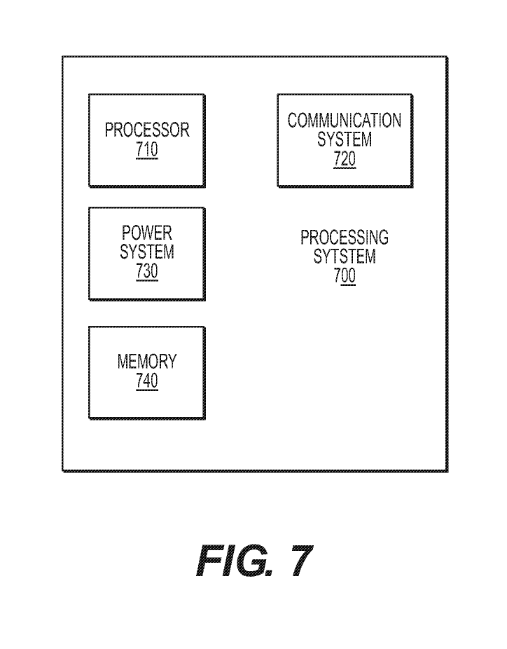

FIG. 7 depicts a schematic of an exemplary processing system 700. Due to the small form factors of the cytometer, system 700 can be implemented as a handheld device, a wearable device, and can be formed as part of the cytometer, or as a separate system in wired or wireless communication with the cytometer. System 700 can include a processor 710, a communication system 720, a power system 730 and a memory 740. Processor 710 can process and/or analyze the acquired video data and perform one or more of the steps described within this disclosure, for example with reference to FIG. 6. Communication system 720 can comprise wired or wireless communication capabilities, including antennas, transceivers, encoders, decoders, etc. In some embodiments, communication system 720 can transmit the processed data to a remote storage device or a remote display device where results can be displayed. In some embodiments, communication system 720 can transmit raw data to a remote processing device or a cloud server, where calculations can be performed and the results can be transmitted to the patient or a healthcare provider. In some embodiments, system 700 can include on-chip electronics to pre-process recorded data prior to processing by processor 710, or prior to transmission to the remote processing device or cloud server. In such embodiments, system 700 can include amplifiers, analog-to-digital converters, multiplexers, and other electronic circuitry to pre-process the acquired data. The power system 730 can power processor 710, communication system 720, and memory 740. In some embodiments, system 700 can be wirelessly powered. In such embodiments, power system 730 can include a supercapacitor, a battery, or some other type of charging system that can be charged wirelessly by a remote device. In some embodiments, optical powering using an array of photovoltaic cells can be used to power the embedded electronics of system 700 or recharge a battery.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. For example, the described implementations include hardware and software, but systems and methods consistent with the present disclosure can be implemented as hardware alone.

Moreover, while illustrative embodiments have been described herein, the scope includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps and/or inserting or deleting steps.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A system for single-file cytometry of the microvasculature of an eye of a subject, the system comprising:
an imaging device including:
imaging optics having at least one lens and a sensor, the lens and the sensor having a focal length permitting cellular resolution of intraocular structures from outside the eye; and
an optics controller coupled to at least one illumination source; and
an image processor coupled to the sensor of the imaging optics, the image processor having a video encoder that creates a microcirculation time sequence showing transit of cells through a microvasculature of the eye.

2. The system of claim 1, wherein the cellular resolution is up to about 10 μm.

3. The system of claim 1, wherein the illumination source provides light at a wavelength between about 540 nm and about 575 nm.

4. The system of claim 3, wherein the illumination source provides light at a wavelength of about 555 nm.

5. The system of claim 1, wherein the illumination source provides light at a wavelength selected from the group consisting of: about 420 nm, about 450 nm, about 586 nm and about 808 nm.

6. The system of claim 1, wherein the lens is a simple objective lens.

7. The system of claim 1, the lens is a telephoto lens.

8. The system of claim 1, wherein the lens is a retrophoto objective lens.

9. The system of claim 1, wherein the sensor is CMOS.

10. The system of claim 1, wherein the sensor is CCD.

11. The system of claim 1, wherein the lens is shaped to optically couple with the lens of the animal eye to image microvasculature within the eye.

12. The system of claim 1, wherein the video encoder is a VP9 encoder.

13. The system of claim 1, wherein the video encoder is an H.264/MPEG-4 HDC video encoder.

14. The system of claim 1, wherein the at least one lens comprises an objective lens and a camera lens.

15. The system of claim 1, wherein the at least one lens comprises an imaging lens.

16. The system of claim 1, wherein the at least one lens comprises an objective lens and a tube lens.

17. The system of claim 1, wherein the at least one lens comprises a liquid lens.

18. The system of claim 1, wherein the imaging optics further comprises a PDAF CMOS chip.

19. A method for eye cytometry, the method comprising:
capturing high-frame-rate video images of a microvasculature within an eye of a subject;
registering the high-frame-rate video images;
filtering the high-frame-rate video images to remove noise and artifacts;
segmenting the high-frame-rate video images; and
creating a microcirculation time sequence from the high-frame-rate video data showing transit of cells through the microvasculature over a period of time.

20. The method of claim 19, further comprising:
calculating the optical density of features within the microcirculation time sequence video; and
calculating the subject's hematocrit from the optical density.

21. The method of claim 19, further comprising:
counting individual cells passing through the microvasculature present in the segmented microcirculation time sequence.

22. The method of claim 19, further comprising:
calculating the flow and volume of blood cells in the segmented microcirculation time sequence video; and
calculating blood flow rate from the flow and volume of blood cells in the segmented microcirculation time sequence video.

23. The method of claim 19, further comprising the steps of:
registering each frame of image data to the previous frame;
calculating the offset of each frame of image data to the previous frame; and
using the offset to calculate eye movement.
